(19) United States
(12) Patent Application Publication
(10) Pub. No.: US 2023/0232959 A1
(43) Pub. Date: Jul. 27, 2023

(54) MANUFACTURING METHOD FOR SEMI-CURED GEL TYPE NAIL

(71) Applicant: LUPIN COMPANY INC., Seoul (KR)

(72) Inventors: Ho Keun Kim, Gyeonggi-do (KR); Hwan Hee Koo, Gyeonggi-do (KR)

(21) Appl. No.: 17/695,503

(22) Filed: Mar. 15, 2022

(30) Foreign Application Priority Data
Jan. 24, 2022 (KR) 10-2022-0009665

(51) Int. Cl. A45D 31/00; B05D 7/00; A61K 8/02; A61Q 3/02

(52) U.S. Cl. CPC A45D 31/00; A61K 8/0233; A61Q 3/02; B05D 7/584; A45D 2200/205

(57) ABSTRACT

The present disclosure discloses a method of manufacturing a semi-cured gel nail for nail art, and more particularly, to a method of manufacturing a UV-C semi-cured nail cured by being irradiated with ultraviolet of a UV-C wavelength. A gel nail according to an embodiment of the present disclosure may have a smooth surface on which luster is maintained and no fingerprint marks or traces remain even when the gel nail comes in contact with a hand or a device.

### CROSS-REFERENCE TO RELATED APPLICATION

This application claims priority of Korean Patent Application No. 10-2022-0009665, filed on Jan. 24, 2022, in the KIPO (Korean Intellectual Property Office), the disclosure of which is incorporated herein entirely by reference.

### BACKGROUND OF THE INVENTION

### Field of the Invention

The present disclosure relates to a method of manufacturing a semi-cured gel nail for nail art, and more particularly, to a method of manufacturing a UV-C semi-cured nail by irradiating ultraviolet of a UV-C wavelength for curing.

### Description of the Related Art

The modern nail beauty industry has shown rapid growth along with the development of the service industry, and today, nail decoration has become a part of fashion and is becoming a necessary condition of daily lives. Nail decoration has become popular regardless as of age and plays an important role in expressing oneself through self-satisfaction or self-realization. According to diversification, individualization, and upgrading, more and more people are creating their own individuality and beauty, and various nail materials and design techniques reflected in the tastes of modern young people always seeking new things are being studied. However, in the case of nail art in which manicure or the like is applied to the nails, this work is very cumbersome and difficult, and a long time is required.

A conventional gel nail has a hard material or is a sticker that is naturally cured quickly when adhered, and thus has problems of poor fitting and low aesthetics.

In order to address these problems, a gel nail sticker such as Korea Patent Registration No. 10-1797203 has been provided. The gel nail sticker is based on a method of attaching a soft gel nail sticker onto the nail and then curing the soft gel nail sticker with UV.

However, although wearing comfort of the soft gel nail sticker is good, when an upper portion of the soft gel nail sticker is touched by a hand or a device, fingerprints, marks, etc. remain, and thus it is difficult to form the surface of a gel nail in a uniform and aesthetic shape.

### SUMMARY OF THE INVENTION

The present disclosure is directed to providing a method of manufacturing a UV-C semi-cured nail, by which a uniform and esthetic surface is formed even when a gel nail comes in contact with a hand or a device.

According to an aspect of the present disclosure, a method of manufacturing a UV-C semi-cured nail includes a release paper forming operation of forming a release paper; an adhesive layer formation operation of forming an adhesive layer onto the release paper; a design layer formation operation of forming a design layer on the adhesive layer; a gel coating operation of forming a soft gel layer on the design layer; a semi-curing operation of semi-curing the soft gel layer; and a UV-C coating layer formation operation of forming a UV-C coating layer by irradiating ultraviolet of a UV-C wavelength onto the soft gel layer.

A gel nail according to an embodiment of the present disclosure has a UV-C coating layer formed on its upper surface, and thus may have a smooth surface on which luster is maintained and no fingerprint marks or traces remain even when the gel nail comes in contact with a hand or a device.

The effects of the present disclosure are not limited to the above-mentioned effects, and other effects not mentioned will be clearly understood by a person skilled in the art from the following description.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a flowchart of a method of manufacturing a UV-C semi-cured nail, according to an embodiment of the present disclosure.

FIG. 2 is a cross-sectional view schematically illustrating a cross-section of a UV-C semi-cured nail according to an embodiment of the present disclosure.

FIG. 3 illustrates a use state of an embodiment of the present disclosure.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

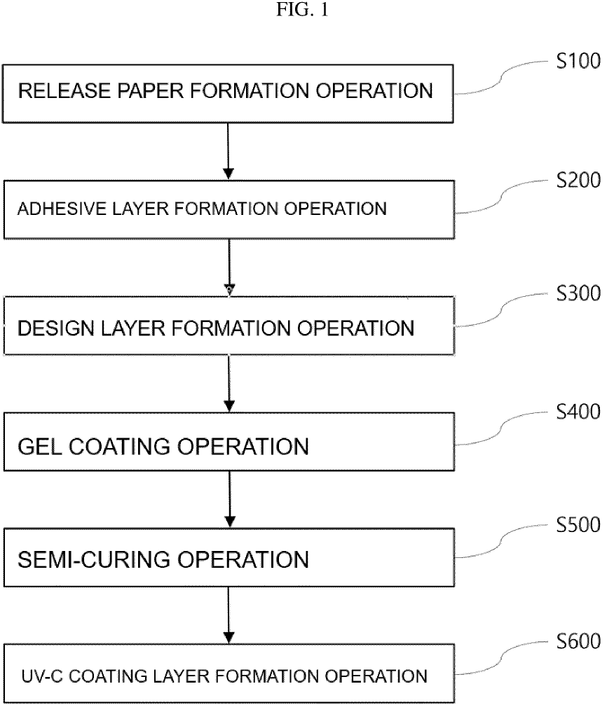

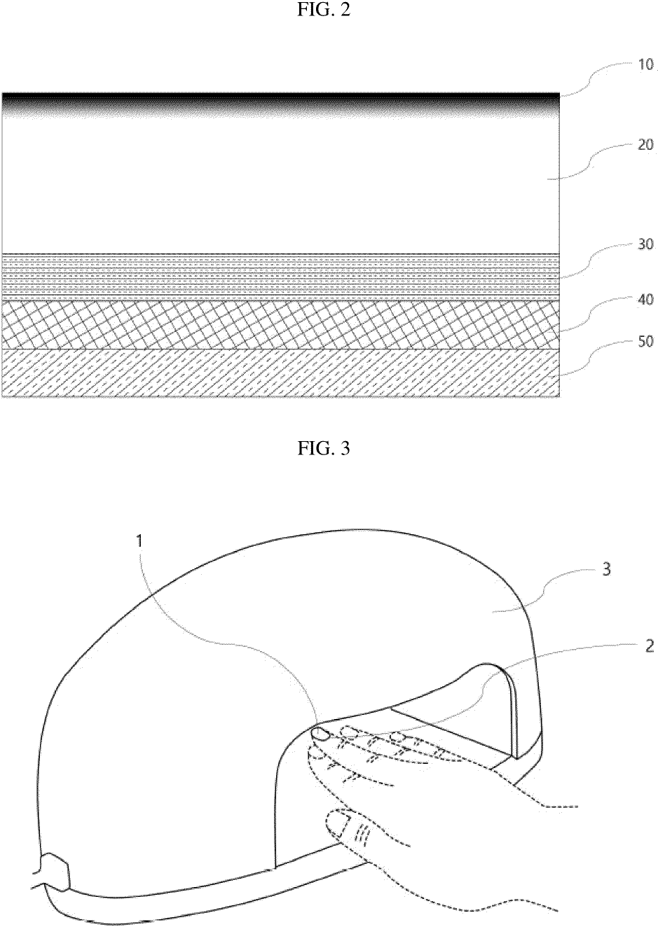

FIG. 1 is a flowchart of a method of manufacturing a UV-C semi-cured nail, according to an embodiment of the present disclosure, FIG. 2 is a cross-sectional view schematically illustrating a cross-section of the UV-C semi-cured nail according to an embodiment of the present disclosure, and FIG. 3 illustrates a use state of an embodiment of the present disclosure.

An embodiment of the present invention is a method of manufacturing a gel nail that is used by being attached and cured to decorate and extend a nail or toenail.

As shown in FIG. 2, a gel nail 1 according to an embodiment of the present disclosure includes a release paper 50, an adhesive layer 40, a design layer 30, a soft gel layer 20, and a UV-C coating layer 10.

The release paper 50 is a portion located below the adhesive layer 40, protects the adhesive layer 40, and is released when being used, and is a paper or a synthetic resin material that is frequently used in a sticker and is detached when the sticker is attached. When the sticker is actually used, the release paper 50 is separated and removed from the sticker.

The adhesive layer 40, which is a portion attached to a nail or a toenail, is adhesive.

The design layer 30, which is a portion located over the adhesive layer 40 and expresses a color and a pattern, expresses a design on a synthetic resin material according to a printing method in a preferred embodiment. However, the design layer 30 may be formed by using a synthetic resin material having a color or by coating or coloring ink on the synthetic resin material.

The soft gel layer 20 is formed of a curable material and is located on the design layer 30. The soft gel layer 20 may be cured when its upper portion is irradiated by light of a UV-C wavelength, and thus may form the UV-C coating layer 10.

According to an embodiment of the present disclosure, the soft gel layer 20 may have a modulus of 0.1 to 25 MPa. When the soft gel layer 20 has a modulus of less than 0.1 MPa, softness of the gel nail 1 is too high, and thus it is difficult to handle the gel nail 1, and there is a problem in that the surface of the gel nail 1 is not formed neatly, such as generation of wrinkles or marks. On the other hand, when the soft gel layer 20 has a modulus of more than 25 MPa, hardness of the gel nail 1 is too high, and thus, when the soft gel layer 20 is attached to a nail of a user, the soft gel layer 20 is not bent along a curved surface of the nail and accordingly does not adhere to the nail.

The UV-C coating layer 10 is a portion that is located over the soft gel layer 20 and protects the soft gel layer 20 and the design layer 30 while providing a unique luster effect and makes a user’s nail visually shiny.

According to an embodiment of the present disclosure, the UV-C coating layer 10 may have a Vickers hardness of 5 to 99. When the UV-C coating layer 10 has a Vickers hardness of less than 5, the UV-C coating layer 10 does not protect the gel nail 1, and the surface of the UV-C coating layer 10 may be damaged while the UV-C coating layer 10 is being attached to the user’s nail. On the other hand, when the UV-C coating layer 10 has a Vickers hardness of more than 99, the gel nail 1 is not smoothly bent, and accordingly does not adhere to a user’s nail or toenail.

A method of manufacturing the UV-C semi-cured nail having the above-described structure will now be described in detail.

Referring to FIG. 1, a method of manufacturing the UV-C semi-cured nail according to an embodiment of the present disclosure includes a release paper formation operation S100, an adhesive layer formation operation S200, a design layer formation operation S300, a gel coating operation S400, a semi-curing operation S500, and a UV-C coating layer formation operation S600.

In the manufacturing method, the release paper 50 is prepared for, and then the adhesive layer 40, the design layer 30, the soft gel layer 20, and the UV-C coating layer 10 are sequentially formed on the release paper 50.

In more detail, the release paper formation operation S100 is an operation of forming the release paper 50. In operation S100 of forming the release paper 50, the release paper 50, which is a basis for forming the gel nail 1, may be prepared for, and the release paper 50 may be formed to have the shape of various nails and toenails of various sizes.

The adhesive layer formation operation S200 is an operation of forming the adhesive layer 40 on the release paper 50. In the adhesive layer formation operation S200, an adhesive is coated on the release paper 50 prepared in the release paper formation operation S100 to thereby form the adhesive layer 40.

The design layer formation operation S300 is an operation of forming the design layer 30 on the adhesive layer 40. In the design layer formation operation S300, a color and a pattern are expressed on the adhesive layer 40 attached in the adhesive layer formation operation S200 to thereby form the design layer 30.

The gel coating operation S400 is an operation of forming the soft gel layer 20 on the design layer 30. In the gel coating operation S400, a gel is coated on the design layer 30 formed in the design layer formation operation S300 to thereby form the soft gel layer 20.

The semi-curing operation S500 is an operation of semi-curing the soft gel layer 20.

In the semi-curing operation S500, various methods such as ultraviolet (UV) radiation and forced blowing using heat may be applied as a method of semi-curing the soft gel layer 20 formed in the gel coating operation S400.

The UV-C coating layer formation operation S600 is an operation of forming the UV-C coating layer 10 by irradiating ultraviolet of a UV-C wavelength onto the soft gel layer 20. In the UV-C coating layer formation operation S600, UV-C ultraviolet is irradiated onto the soft gel layer 20 coated in the gel coating operation S400. Because the UV-C coating layer 10 is a result of curing an upper portion of the soft gel layer 20, a boundary surface between the UV-C coating layer 10 and the soft gel layer 20 may be continuous.

According to an embodiment of the present disclosure, the wavelength of the ultraviolet irradiated in the UV-C coating layer formation operation S600 may be 100 to 250 nm. When ultraviolet with a wavelength of less than 100 nm is irradiated in the UV-C coating layer formation operation S600, the UV-C coating layer 10 has a very small thickness due to a low transmittance of the ultraviolet, and thus cannot protect the soft gel layer 20, and marks or traces are generated during attachment. On the other hand, when ultraviolet with a wavelength of more than 250 nm is irradiated in the UV-C coating layer formation operation S600, the UV-C coating layer 10 is too thick to be smoothly bent, and thus does not adhere to a nail.

According to an embodiment of the present disclosure, the brightness of the ultraviolet irradiated in the UV-C coating layer formation operation S600 may be 10 to 1000 lm. When ultraviolet with a brightness of less than 10 lm is irradiated in the UV-C coating layer formation operation S600, the UV-C coating layer 10 has a low hardness and accordingly is damaged. On the other hand, when ultraviolet with a brightness of more than 1000 lm is irradiated in the UV-C coating layer formation operation S600, the UV-C coating layer 10 is too hard, and thus the gel nail 1 is not smoothly bent and accordingly does not adhere to a nail or toenail.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A method of manufacturing a UV-C semi-cured gel nail in a gel nail that is used by being attached and cured to decorate and extend a nail or toenail, the gel nail comprising a release paper, an adhesive layer, a design layer, a soft gel layer, and a UV-C coating layer, wherein the soft gel layer is formed of a curable material, the method comprising:
a release paper forming operation of forming a release paper;
an adhesive layer formation operation of forming an adhesive layer on the release paper;
a design layer formation operation of forming a design layer on the adhesive layer;
a gel coating operation of forming a soft gel layer on the design layer;
a semi-curing operation of semi-curing the soft gel layer; and
a UV-C coating layer formation operation of forming a UV-C coating layer by irradiating ultraviolet of a UV-C wavelength onto the soft gel layer.

2. The method of claim 1, wherein a wavelength of the ultraviolet irradiated in the UV-C coating layer formation operation is 100 to 250 nm.

3. The method of claim 1, wherein a brightness of the ultraviolet irradiated in the UV-C coating layer formation operation is 10 to 1000 lm.

4. The method of claim 1, wherein the soft gel layer has a modulus of 0.1 to 25 MPa.

5. The method of claim 1, wherein the UV-C coating layer has a Vickers hardness of 5 to 99.

6. The method of claim 1, wherein the design layer is formed by using a synthetic resin material.

7. A method of manufacturing a UV-C semi-cured gel nail in a gel nail that is used by being attached and cured to decorate and extend a nail or toenail, the method comprising:
forming a release paper;
forming an adhesive layer on the release paper;
forming a design layer on the adhesive layer;
forming a soft gel layer on the design layer, the soft gel layer is formed of a curable material;
semi-curing the soft gel layer; and
forming a UV-C coating layer by irradiating ultraviolet of a UV-C wavelength onto the soft gel layer.

8. The method of claim 7, wherein a wavelength of the ultraviolet irradiated in the UV-C coating layer formation operation is 100 to 250 nm.

9. The method of claim 7, wherein a brightness of the ultraviolet irradiated in the UV-C coating layer formation operation is 10 to 1000 lm.

10. The method of claim 7, wherein the soft gel layer has a modulus of 0.1 to 25 MPa.

11. The method of claim 7, wherein the UV-C coating layer has a Vickers hardness of 5 to 99.

12. The method of claim 7, wherein the design layer is formed by using a synthetic resin material.
